(19) United States
(12) Patent Application Publication
(10) Pub. No.: US 2004/0010278 A1
(43) Pub. Date: Jan. 15, 2004

(54) Surgical knife

(76) Inventors: Shoichi Nakamura, Saitama (JP); Nagahisa Machida, Saitama (JP)

Correspondence Address: Kanesaka & Takeuchi, Suite 2, 1423 Powhatan Street, Alexandria, VA, 22314, US

(21) Appl. No.: 10/415,368

(22) PCT Filed: Dec. 4, 2000
(86) PCT No.: PCT/JP00/08574

(51) Int. Cl. A61B 17/32

(52) U.S. Cl. 606/170

(57) ABSTRACT

A surgical knife which is formed with air vent holes in the vicinity of the cutting edge of the blade to provide improved sharpness during the incision without the possibility of incised living tissue sticking to the blade surface and improved visibility of the incised surface or cut region.

The surgical knife has a stem and a cutting edge, wherein one or a plurality of through-holes extending from the front to the back to serve as air vent holes during the incision of living tissue are formed in the vicinity of the cutting edge surface formed on the blade. The through-holes are not limited in shape to a circle; they may be oval or polygonal or in the form of slits or other elongated openings. They may also be a plurality of very small holes.

### TECHNICAL FIELD

This invention relates to a surgical knife (scalpel) used as medical instruments for the human body and domestic animals.

### BACKGROUND

Generally, a knife used in surgical operation comprises a cutting edge and a stem, and those different in shape of the cutting edge by use are hundreds of kinds or, thousands of kinds or more when the size is taken into consideration.

For a knife for incision, it is necessary to form a cutting edge having a blade capable of transmitting feeling at the time of incision to the doctor for performing operation as much as possible in order to prepare an accurate opening. However, one that is satisfied with this demand could not have been realized.

Further, in the knife for incision, the incised surface or cut region is seen by the doctor for performing operation as much as possible in order that living tissue of an incised region is opened, and deeper incision more than as needed is not made. However, one that is satisfied with this demand could not have been realized heretofore.

### DISCLOSURE OF THE INVENTION

It is an object of the present invention to provide a surgical knife in which air vent holes are provided in the vicinity of a cutting edge of a blade whereby at the time of incision of living tissue, both rigidity and sharp sharpness are provided, and the air vent holes have a part of a incision depth graduation.

It is a further object of the invention to provide a surgical knife which has improved visibility of the incised surface or cut region at the time of incision without sticking the incised living tissue to the blade surface by the air vent holes formed in the blade.

In the surgical knife according to the present invention, through-holes extending from the front to the back are provided in the vicinity of the cutting edge surface formed in the blade of the cutting edge.

Further, in the surgical knife according to the present invention, through-holes extending from the front to the back along the cutting edge surface are provided in the vicinity of the cutting edge surface formed in the blade of the cutting edge.

The through-holes may be elongated slits or very small holes, and the shape of the though-holes may be circular, oval, triangular, square, polygonal, or radially disposed slits.

Furthermore, in the surgical knife according to the present invention, through-holes extending from the front to the back are provided in the vicinity of the cutting edge surface formed in the blade of the cutting edge, and the cutting edge can be removed from the stem and exchanged. And, mounting of the cutting edge to the stem is done by making use of engagement of threads or elasticity of a spring or collet chuck.

Further, a substance having a water-repelling property is coated on the blade.

### BRIEF DESCRIPTION OF THE DRAWINGS

FIG. 1 is a view showing a first embodiment of a surgical knife according to the present invention.

FIG. 2 is a view showing a cutting edge of FIG. 1.

FIG. 3 is an embodiment in which one circular through-hole is provided in an extreme end of a blade.

FIG. 4 is an embodiment in which two oval through-holes are provided in an extreme end of a blade.

FIG. 5 is an embodiment in which a row of through-holes are provided along the cutting edge surface.

FIG. 6 is an embodiment in which a number of very small holes are provided in a portion of a blade provided with the cutting edge surface.

FIG. 7 is a view showing a second embodiment of a surgical knife according to the present invention.

FIG. 8 is a view showing a cutting edge of FIG. 7.

FIG. 9 is an embodiment in which radially disposed slits are provided in an extreme end of a blade.

FIG. 10 is a third embodiment in which a cutting edge is mounted on a stem by a collet chuck.

FIG. 11 is an embodiment in which through-holes are provided along the cutting edge surface, and a substance having a water-repelling property is coated on the extreme end of a blade.

### BEST MODE FOR CARRYING OUT THE INVENTION

The embodiment of the present invention will be described hereinafter with reference drawings. Parts having the same reference numeral as those in the respective drawings represent similar parts, and detailed description thereof will be omitted.

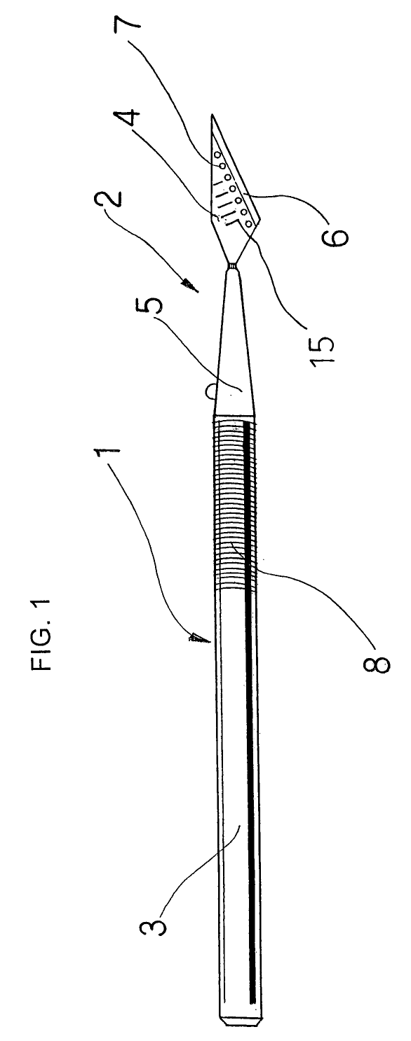

FIG. 1 is a view showing a first embodiment of a surgical knife according to the present invention. A cutting edge 2 is detachably secured to a stem 3 of a knife 1. The cutting edge 2 is formed such that a blade 4 and a mounting portion 5 to the stem 3 are integrated.

The blade 4 is worked into the desired shape by precisely pressing steel capable of providing both rigidity and sharp sharp-ness, for example, molybdenum vanadium steel. And, burr caused by applying electrolytic polishing and pressing to the blade 4 is removed to thereby make mounting it on an exclusive-use fixture positive.

Next, polishing work is carried out using the exclusive-use fixture. At this time, for keeping the same sharpness always with respect to a complicated knife shape such as R, an NC controlled polishing machine is used so that the grinding direction is at a fixed angle from the R center, which is interlocked with the exclusive-use fixture to carry out working for a suitable shape. Thereafter, in the state of being remained secured to the exclusive-use fixture, one or a plurality (not less than two) of rows of through-holes 7 from the surface to the back of the blade 4 are bored along the cutting edge surface 6 in the vicinity of the cutting edge surface 6 by laser beam working, discharge working, plastic working (punch press) or the like.

Further suitable very small shapes (for example, dimple shape, slit shape or the like) are worked on the surface using laser beam working, discharge working, ion edging working or the like). Thereafter, using a few kinds of grindstones, finish-working is carried out using the exclusive-use fixture similarly to that mentioned above.

As the final working process, for adjusting the sharpness of the knife to the using characteristic, fine burr stuck to the cutting edge is again removed by the electrolytic polishing. A combination of the through-holes 7, fine working of the surface, and two kinds of very small shapes (for example, such as dimple shape, slit shape or the like) serves as vent holes of air, body fluids (blood), fat or the like during the incision of living tissue, and there is no possibility of incised living tissue sticking to the blade 4.

Lately, for preventing contagion of diseases resulting from the contact with blood contaminated, it is general that the cutting edge 2 is used only once. Therefore, in the embodiment, the cutting edge 2 is made exchangeable with respect to the stem 3. The stem 3 may be made of metal or plastic, but a nonskid 8 is provided so that the knife 1 may be gripped securely during the operation.

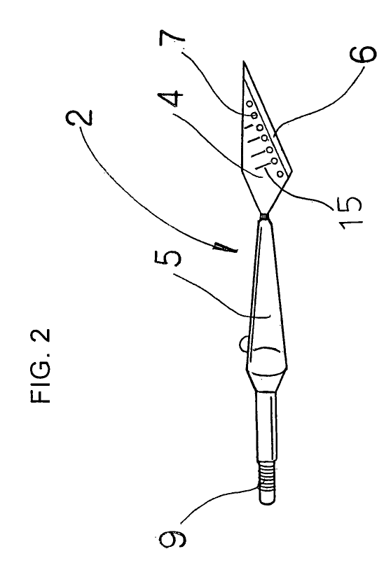

FIG. 2 is a view showing the cutting edge 2. The blade 4 is secured to and integrated with the mounting portion 5. The mounting portion 5 is formed with an external thread 9, and the cutting edge 2 is engaged with the internal thread (not shown) formed on the stem 3, and is mounted detachably. The fact that the cutting edge is mounted on the stem by engagement of threads as described produces no play caused by mounting, which is therefore suitable for the surgical knife whose blade is small for carrying out precise operation.

As shown in FIGS. 1 to 3, marks 15 showing the distance from the extreme end of the blade may be notched in the surface or the back of the blade. Incidentally, the marks 15 may be used both as the through-holes or slits provided in the vicinity of the cutting edge surface according to the present invention. By the marks 15 as described, an aim of incision depth of the skin can be known easily during operation. Needless to say, the marks 15 showing the distance from the extreme end of the blade can be similarly applied to the other embodiments of the present invention.

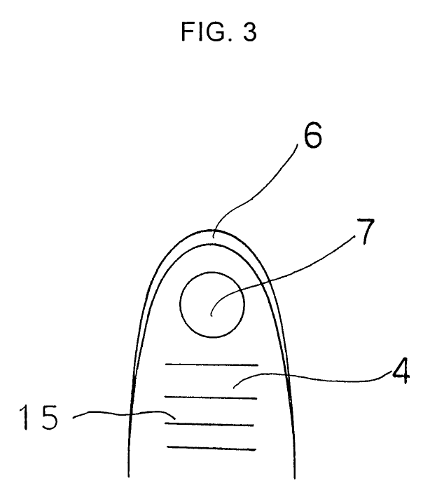

FIGS. 3 to 6 and 9 are respectively views showing the extreme end of the blade.

In the embodiment shown in FIG. 3, in the vicinity of the cutting edge surface 6 in the form of a circular are provided at the extreme end of the blade 4 is bored one circular through-hole 7 from the surface to the back of the blade 4 adjusting to the shape thereof. At the time of incision, living tissue is proceeded to cut in a taper of the cutting edge surface 6, and an opening is opened by the presence of the through-hole 7 serving as the thickness of the blade and the air vent hole, and the incised surface is not stuck to the surface of the blade 4. Thereby, since the vicinity of the extreme end of the blade surface 4 can be visualized, incision operation is performed easily for a surgeon.

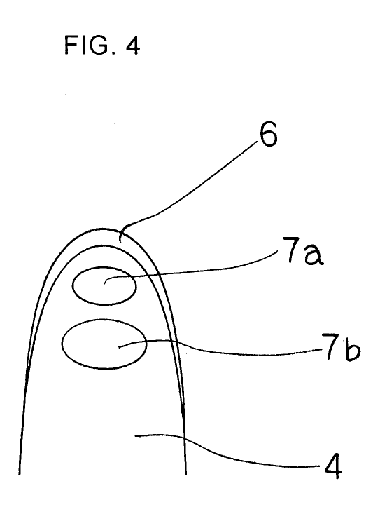

In the embodiment shown in FIG. 4, in the vicinity of the cutting edge surface 6 in the form of a circular arc provided at the extreme end of the blade 4 are bored, adjusting to the shape thereof, a small oval through-hole 7a close to the extreme end and an oval through-hole 7b larger than the former adjacent thereto. In this embodiment, the effect similar to that of the embodiment shown in FIG. 3 is obtained even though feeling at the time of incision is delicately different. It is noted that the number of oval holes is not limit to two, but a plurality (more than three) of holes may be provided.

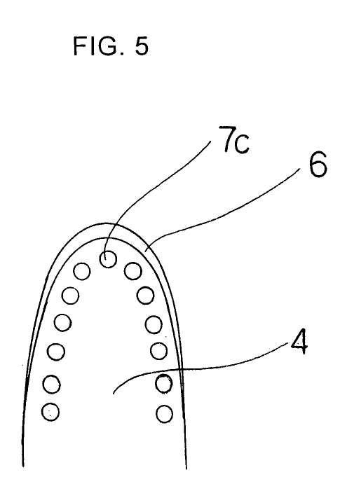

In the embodiment shown in FIG. 5, in the vicinity of the cutting edge surface 6 are provided at the extreme end of the blade 4 are bored, along the cutting edge surface 6, a plurality of circular through-holes 7c having the radius about ⅕ to 1/10 as compared with the radius of curvature of the cutting edge surface 6.

Also in this embodiment, the similar effect is obtained even though feeling at the time of incision is delicately different from the other embodiments.

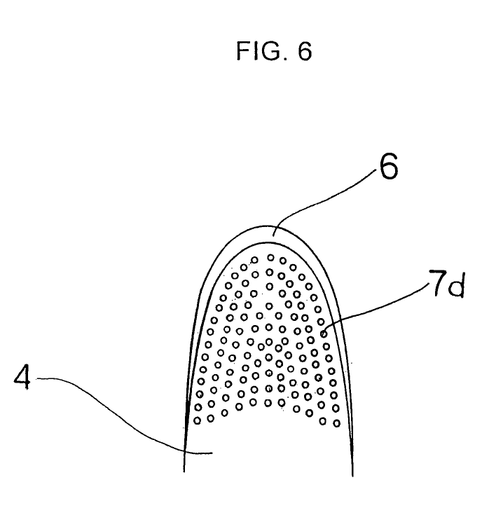

In the embodiment shown in FIG. 6, a number of small through-holes 7d are bored in the blade surface 4 in the vicinity of a portion in which the cutting edge surface 6 is provided. Also in this embodiment, the similar effect is obtained even though feeling at the time of incision is somewhat different from the other embodiments.

The through-hole 7 bored in the blade 4 need not be circular, but an oval type, a triangular shape, a square type, a polygonal shape, a star type or the like may be employed.

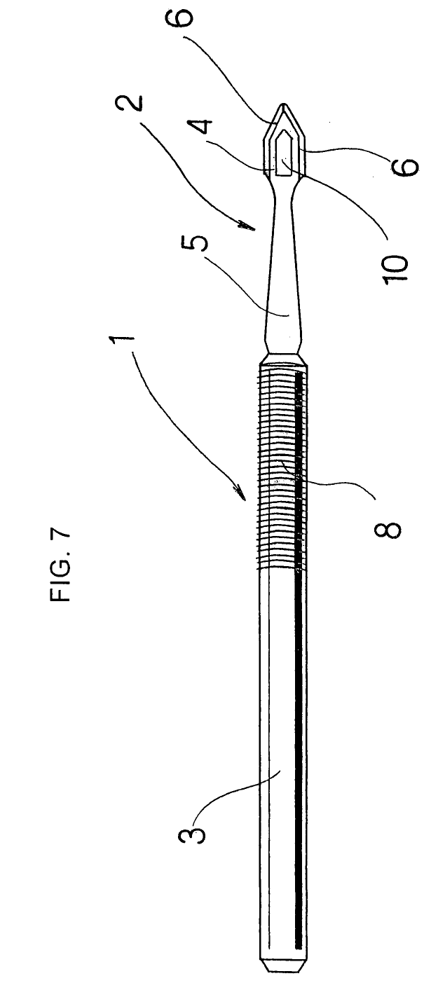

FIG. 7 is a view showing a second embodiment of a surgical knife according to the present invention. A cutting edge 2 is detachably secured to a stem 3 of a knife 1. In the cutting edge 2, a blade 4 and a mounting portion 5 are integrally formed from a material similar to that of the first embodiment and in manufacturing process similar thereto.

The peripheral edge of the blade 4 is polished, and the cutting edge surface 6 is formed taper-like. In the blade 4, an elongated slit 10 is formed extending through from the surface to the back surface in the vicinity of the cutting edge surface 6. The number of the slit 10 is not limited to one, but even if a plurality of them are formed, the similar effect can be obtained.

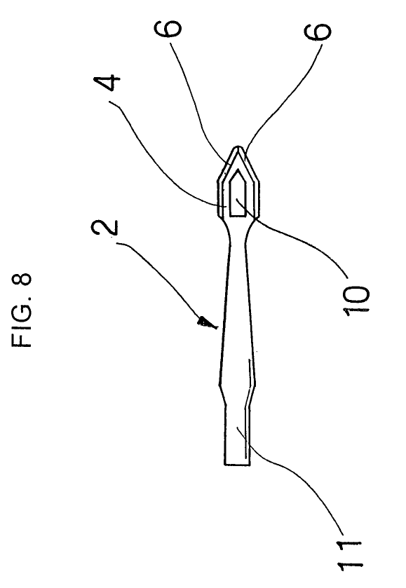

FIG. 8 is a view showing the cutting edge 2. An insert portion 11 provided on the other end of the blade 4 is inserted into the stem 3, and is detachably mounted on the stem 3 elastically by means of a spring or the like in a well known method (not shown).

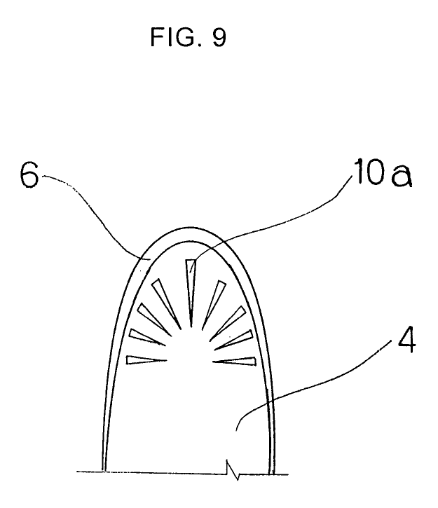

In the embodiment shown in FIG. 9, slits 10a are arranged radially extending through from the surface to the back surface in the vicinity of the extreme end of the blade 4 in which the cutting edge surface 6 is provided.

Needless to say, the surgical knives mentioned in the foregoing embodiments can be utilized for surgical operation for not only the human body but also animals such as domestic animals or pets.

Materials of the mounting portion 5 need not be particularly limited if they can secure the blade 4 to the mounting portion 5 securely. It is also possible to employ a structure in which the blade 4 is detachably mounted on the stem 3 directly without intervention of the mounting portion 5.

Further, without employing the structure capable of replacing the cutting edge 2 with respect to the stem 3, both may be integrated.

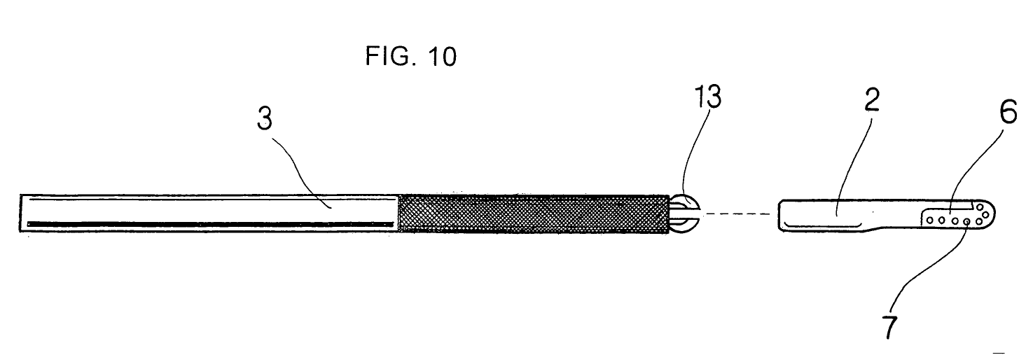

FIG. 10 is a view showing a third embodiment in which the cutting edge 2 is mounted on the stem 3 by a collet chuck 13. The cutting edge surface 6 of the cutting edge 2 is bored with a plurality of through-holes 7. The cutting edge 2 may be any cutting edge surface if it can be secured by the collet chuck 13.

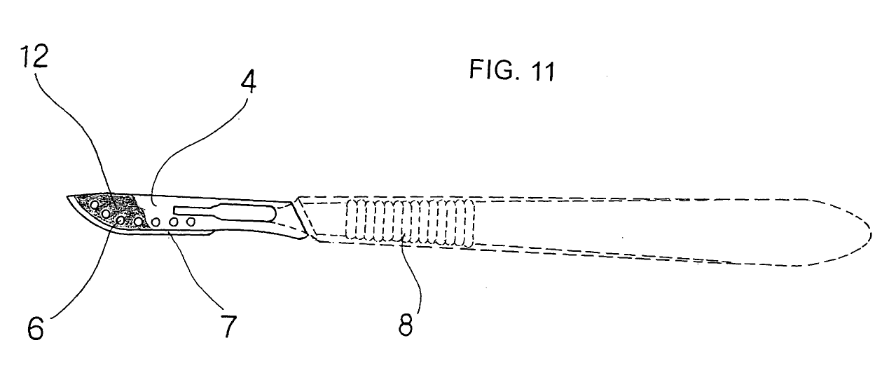

The embodiment of FIG. 11 is basically the same as the embodiment of FIG. 1 in that a plurality of through-holes 7 are bored along the cutting edge surface 6 of the blade 4. However, a substance 12 having a water-repelling property is coated in the vicinity of the extreme end of the blade 4. As the substance having a water-repelling property, synthetic resin, paraffin, casein, soybean protein, wax or the like is suitable. In this embodiment, out of these substances, one is selected for coating.

With this, since both oil and water are repelled during incision operation, visibility of incised surface or cut part is improved to facilitate incision operation.

According to the present invention, the through-holes as air vent holes are provided in the vicinity of the cutting edge surface of the blade, the opening of the incised part becomes sharp and is opened naturally, and moreover, the air vent hole serves as a graduation of depth for incision at a minimum as necessary. Therefore, the cut living tissue is not stuck to the blade surface, and the incision operation can be performed easily.

Further, since the cut surface of living tissue is not broken, anastomosis is easy, and a wound is recovered quickly.

Furthermore, since the incised surface is opened clearly, visibility is improved, operation can be performed maintaining accurate incised depth, and a lower blood vessel or the like is not wounded.

1. A surgical knife having a stem and a cutting edge, characterized in that through-hole(s) extending from the front to the back are provided in the vicinity of a cutting edge surface formed in a blade of said cutting edge.

2. A surgical knife having a stein and a cutting edge, characterized in that a plurality of through-holes extending from the front to the back of said blade along said cutting edge surface are provided in the vicinity of the cutting edge surface formed in a blade of said cutting edge.

3. The surgical knife according to claim 1 or 2, wherein said through-hole is an elongated slit extending from the front to the back of said blade.

4. The surgical knife according to claim 1 or 2, wherein a substance having a water-repelling property is coated on said blade.

5. The surgical knife according to claim 1, wherein said through-holes are a plurality of very small holes.

6. The surgical knife according to claim 1 or 2, wherein the shape of said through-hole(s) is circular, oval, triangular, square, polygonal, elongated slits or radially disposed slits.

7. The surgical knife according to claim 6, wherein marks showing the distance from an extreme end of the blade are notched in the front or the back of said blade.

8. A surgical knife having a stein and a cutting edge, characterized in that through-hole(s) extending from the front to the back are provided in the vicinity of the cutting edge surface formed in a blade of said cutting edge, and said cutting edge can be removed from said stem and replaced.

9. The surgical knife according to claim 8, wherein mounting of said cutting edge to said stem is performed by engagement of threads.

10. The surgical knife according to claim 9, wherein mounting of said cutting edge to said stem is performed by making use of elasticity of a spring.

11. The surgical knife according to claims 8 and 9, wherein mounting of said cutting edge to said stem is performed by making use of a collet chuck.
